SEQUENCE LISTING (11 sequences)

SEQ ID NO: 1 - DNA, 2537 bp, Homo sapiens
  CDS (842)..(2011)
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggcca

SEQ ID NO: 2 - protein, 390 aa, Homo sapiens [sequence omitted]

SEQ ID NO: 3 - DNA, 2745 bp, Homo sapiens
  CDS (842)..(2014)
acctccctccgcggagcagccagacagcgagggccccggccgggggcaggggggacgccccgtccggggcaccccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggagcctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggcccgccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacaccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatggcagggactctgataacacccattttaaaggttgaggaaacaagcccagagaggttaagggaggagttcctgcccaccaggaacctgctttagtgggggatagtgaagaagacaataaaagatagtagttcaggccaggcggggtgctcacgcctgtaatcctagcacttttgggaggcagagatgggaggatacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 4 - protein, 391 aa, Homo sapiens
MPPSGLRLLPLLLPLLWLLVLTPGPPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 5 - DNA, 1695 bp, Homo sapiens
caagcaggatacgtttttctgttgggcattgactagattgtttgcaaaagtttcgcatcaaaaacaaacaacaacaacaaaaaaccaaacaactctccttgatctatactttgagaattgttgatttctttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaatttttttaaa

SEQ ID NO: 6 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 7 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 8 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 9 - DNA, 2570 bp, Homo sapiens
gcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcag

SEQ ID NO: 10 - DNA, 2574 bp, Homo sapiens
cctgtttagacacatggacaacaatcccagcgctacaaggcacacagtccgcttcttcgtcctcagggttgccagcgcttcctggaagtcctgaagctctcgcagtgcagtgagttcatgcaccttcttgccaagcctcagtctttgggatctggggaggccgcctggttttcctccctccttctgcacgtctgctggggtctcttcctctccaggccttgccgtccccctggcctctcttcccagctcacacatgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctgagaccccacgtgcgacagagagaggggagagagaaccaccactgcctgactgcccgctcctcgggaaacacacaagcaacaaacctcactgagaggcctggagcccacaaccttcggctccgggcaaatggctgagatggaggtttccttttggaacatttctttcttgctggctctgagaatcacggtggtaaagaaagtgtgggtttggttagaggaaggctgaactcttcagaacacacagactttctgtgacgcagacagaggggatggggatagaggaaagggatggtaagttgagatgttgtgtggcaatgggatttgggctaccctaaagggagaaggaagggcagagaatggctgggtcagggccagactggaagacacttcagatctgaggttggatttgctcattgctgtaccacatctgctctagggaatctggattatgttatacaaggcaagcatttttttttttaaagacaggttacgaagacaaagtcccagaattgtatctcatactgtctgggattaagggcaaatctattacttttgcaaactgtcctctacatcaattaacatcgtgggtcactacagggagaaaatccaggtcatgcagttcctggcccatcaactgtattgggccttttggatatgctgaacgcagaagaaagggtggaaatcaaccctctcctgtctgccctctgggtccctcctctcacctctccctcgatcatatttccccttggacacttggttagacgccttccaggtcaggatgcacatttctggattgtggttccatgcagccttggggcattatgggtcttcccccacttcccctccaagaccctgtgttcatttggtgttcctggaagcaggtgctacaacatgtgaggcattcggggaagctgcacatgtgccacacagtgacttggccccagacgcatagactgaggtataaagacaagtatgaatattactctcaaaatctttgtataaataaatatttttggggcatcctggatgatttcatcttctggaatattgtttctagaacagtaaaagccttattctaaggtg

SEQ ID NO: 11 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

(19) United States
(12) Patent Application Publication
(10) Pub. No.: US 2005/0164909 A1
(43) Pub. Date: Jul. 28, 2005

(54) Methods and reagents for treating inflammation and fibrosis

(75) Inventors: Clay B. Marsh, Columbus, OH (US); Charles G. Orosz, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 10/892,997

(22) Filed: Jul. 16, 2004

Related U.S. Application Data
(60) Provisional application No. 60/487,826, filed on Jul. 16, 2003.

(51) Int. Cl. A61K 38/17

(52) U.S. Cl. 514/2

(57) ABSTRACT

Methods are provided for providing anti-inflammatory activity and inhibiting a fibrotic disease, such as pulmonary fibrosis, in an individual. The methods comprise administering a biologically effective amount of latency-associated peptide (LAP) to the individual. Also provided are pharmaceutical compositions comprising LAP for use in accordance with these methods.

### PRIORITY CLAIM

This application claims priority to U.S. Provisional Patent Application No. 60/487,826, filed Jul. 16, 2003, which is incorporated herein by reference in its entirety.

### GOVERNMENT RIGHTS

This invention was developed, at least in part, with government support under National Institutes of Health Grants No: RO1 HL 63800, 66108, and PO1 HL 70294. The U.S. government may have certain rights in the invention.

### FIELD OF THE INVENTION

The invention relates to methods for reducing inflammation in an individual. The invention also relates to methods for treating individuals with idiopathic pulmonary fibrosis and other fibrotic diseases, particularly those associated with enhanced inflammation.

### BACKGROUND

Diseases involving inflammation are characterized by an influx of cells into an affected area, secretion of various protein factors, and subsequent tissue irritation and damage. Inflammation is observed in pathologies which include, but are not limited to, chronic obstructive pulmonary diseases of the airways, asthma, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumotitis, occupational asthma, sarcoid, reactive airway disease syndrome, interstitial lung disease, hyper-eosinophilic syndrome, rhinitis, sinusitis, and parasitic lung disease. These various pathologies can be provoked by a variety of inflammatory agents, such as, allergens, cold air, exercise, infections and air pollution. These inflammatory agents stimulate the release of inflammatory mediators that recruit cells involved in inflammation to the affected area. Such cells include lymphocytes, eosinophils, mast cells, basophils, neutrophils, macrophages, monocytes, fibroblasts and platelets.

Left untreated, inflammation can cause serious tissue damage and possibly death in the affected individual. Cellular activity associated with inflammation can lead to development of fibrotic diseases that are characterized by excess production of extracellular matrix, a fibrous material. The presence of this excess fibrous material leads to changes in tissue architecture; tissue architectural changes in organs such as the lung can impair function and lead to severe health effects. Pulmonary fibrosis, idiopathic pulmonary fibrosis, cirrhosis, sarcoidosis, keloids, renal fibrosis (e.g., diabetic nephropathy), retinopathy, organ transplant rejection, atherosclerosis, glomerulonephritis with scarring, cirrhosis, and other conditions with a fibrotic component, are just some examples of diseases related to abnormal accumulation of fibrous material in tissues.

Idiopathic pulmonary fibrosis (IPF), one type of fibrotic disease, is an idiopathic interstitial pneumonia occurring in adults, characterized by dyspnea, hypoxia, diffuse pulmonary infiltrates and usual interstitial pneumonia (UIP). Destruction and fibrosis of the lung interstitium and parenchyma predominate in the disease. Median survival of afflicted individuals is approximately 4-5 years after onset of symptoms.

Conventional treatments for inflammation consist of various anti-inflammatory therapies. However, these treatments are not very effective. Moreover, the majority of anti-inflammatory and symptomatic relief reagents cause serious side effects, which include, but are not limited to, increased susceptibility to infection, liver toxicity, drug-induced lung disease, and bone marrow suppression. In many instances, the benefits of treatment are outweighed by the harm caused by the treatment. Thus, there is a requirement for less harmful and more effective reagents for treating inflammatory activity. Likewise, there is a requirement for additional agents for treating and preventing fibrotic pathologies that may be attendant to inflammation in an affected individual.

### SUMMARY OF THE INVENTION

The present invention provides methods for reducing or controlling inflammation in an individual such as a mammalian subject. The method comprises administering a biologically effective amount of latency-associated peptide (LAP) to a subject in need of the same. LAP is a peptide that in some embodiments is processed from a precursor protein that also contains TGF-β. Advantageously, LAP reduces inflammatory activity in mammalian subjects without causing fibrosis.

The present invention also provides methods for inhibiting or controlling development or progression of a fibrotic disease in a mammalian subject. The method comprises administration of LAP to the subject. The invention is based, at least in part, on applicants' finding that LAP also interferes with the processes involved in fibrosis; specifically, LAP interferes with the profibrotic activities of TGF-β by a mechanism that is independent of LAP binding to TGF-β. Thus, in another aspect, the invention also provides methods for preventing or treating fibrosis.

The present invention also provides pharmaceutical compositions containing LAP for use in the above methods.

The methods and compositions of the present invention are useful for reducing or controlling inflammation or fibrosis in an individual suffering from, or at risk of developing, pathologies that include, but are not limited to, fibrotic diseases and conditions, such as pulmonary fibrosis, idiopathic pulmonary fibrosis, cirrhosis, sarcoidosis, keloids, renal fibrosis (e.g., diabetic nephropathy), retinopathy, organ transplant rejection, atherosclerosis, glomerulonephritis with scarring, cirrhosis, ARDS, fibrotic cancer, fibrosis of the lungs, arteriosclerosis, post myocardial infarction, cardiac fibrosis, post-angioplasty restenosis, renal interstitial fibrosis, scarring and diabetes-associated pathologies, and other conditions with a fibrotic component. According to the present invention, tissues including the lung, kidney, liver, and skin may be targeted, either by systemic or local administration of various forms of LAP, to treat an individual suffering from or at risk of developing pathologies involving inflammation or fibrosis.

Additional features and advantages of the invention will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention, and together with the description, serve to explain the principles of the invention.

### BRIEF DESCRIPTION OF THE DRAWINGS

The present invention may be more readily understood by reference to the following drawings wherein:

FIG. 1 shows a schematic of LAP and TGF-β in free (upper and lower panels) and latent-complex bound forms (middle panel).

FIG. 2 shows the nucleotide sequence and predicted amino acid sequence of human TGF-β1 (precursor protein), and corresponds to FIG. 2B from U.S. Pat. No. 5,801,231.

FIG. 3 shows another nucleotide sequence encoding human TGF-β1 and LAP, which sequence corresponds to that reported in GenBank under Accession No. X02812 J05114.

FIG. 4 shows another nucleotide sequence encoding human TGF-β1 and its associated LAP.

FIG. 5 shows a nucleotide sequence encoding human TGF-β2 and its associated LAP homolog.

FIG. 6 shows another nucleotide sequence encoding human TGF-β2 and its associated LAP homolog, which sequence corresponds to that reported in GenBank under Accession Nos. M19154, M22045, and M22046.

FIG. 7 shows a nucleotide sequence encoding human TGF-β3 and its associated LAP homolog, which sequence corresponds to that reported in GenBank under Accession No. NM-003239

FIG. 8 shows the results of a study in which LAP has induced a reduction in delayed type hypersensitivity reaction (DTHR).

FIG. 9. shows the results of a study in which LAP has resulted in inhibition of TGF-β stimulation of plasminogen activator inhibitor-1 promoter, wherein such action of LAP is by a mechanism other than binding to TGF-β.

FIG. 10 shows the results of a study in which LAP has resulted in inhibition of TGF-β-dependent production of hydroxyproline and collagen.

### DETAILED DESCRIPTION OF THE INVENTION

TGF-β Activities and Isoforms

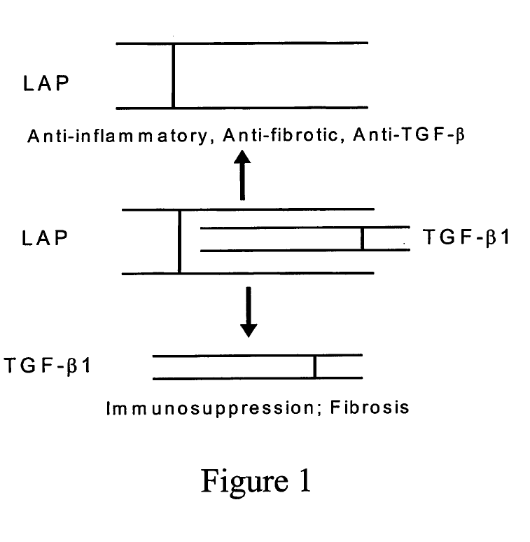

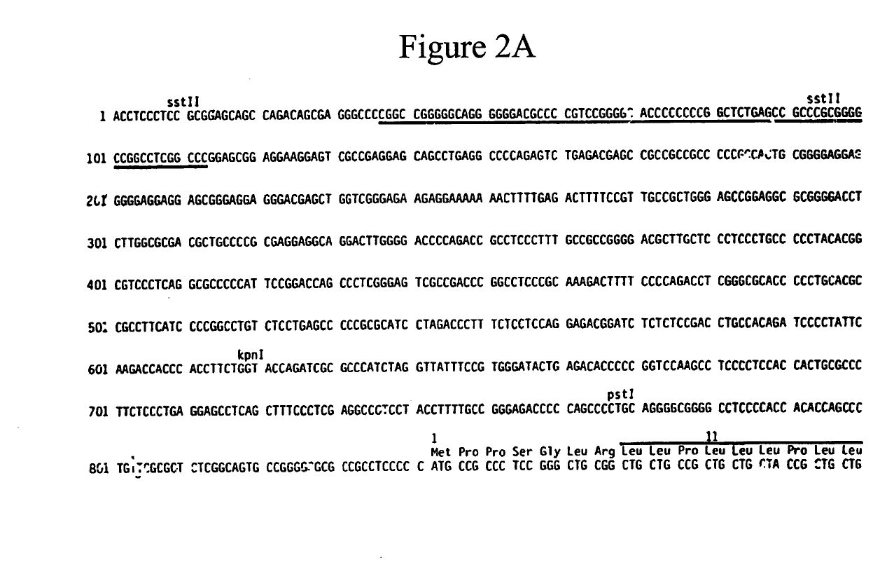

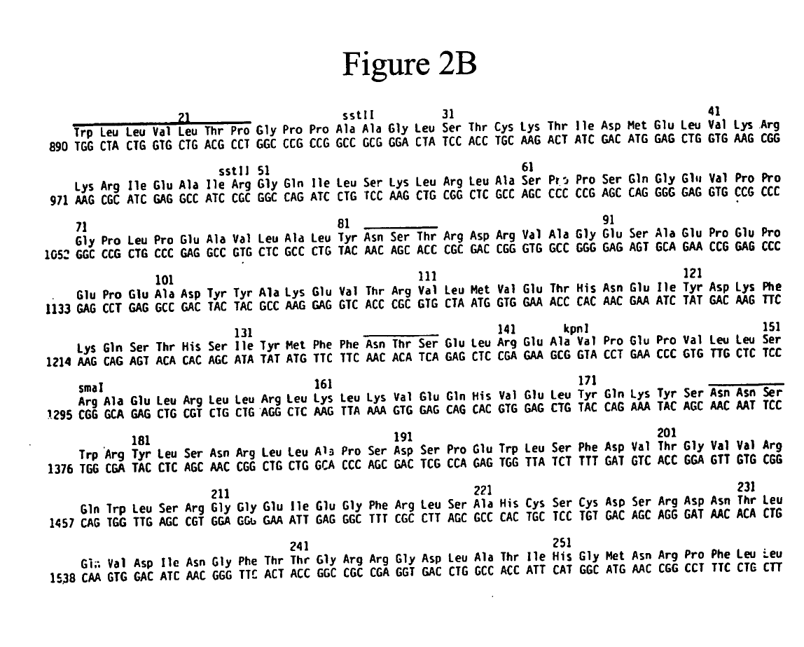

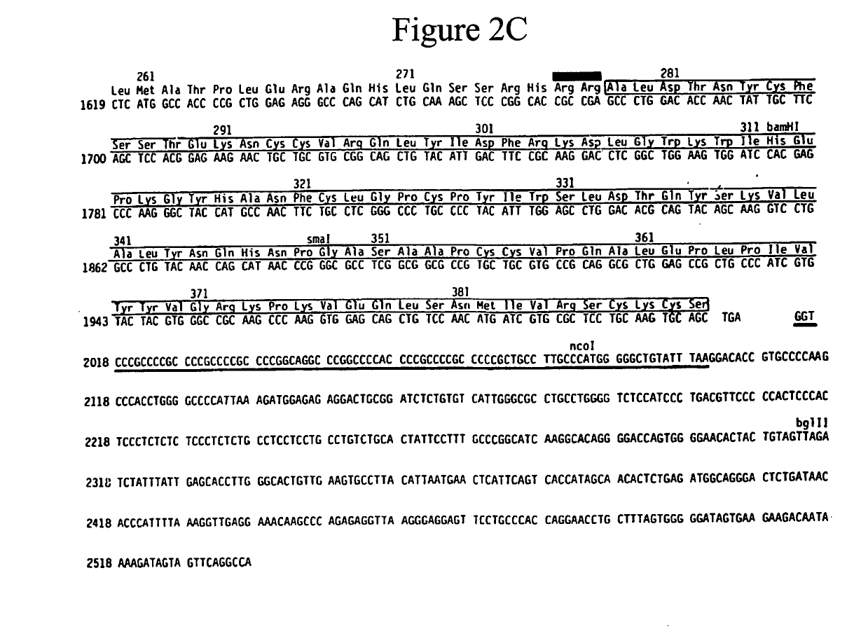

There are multiple isoforms of transforming growth factor beta (TGF-β), including TGF-β1, 2, 3, 4, and 5. This family of TGF-β isoforms constitutes a group of cytokines which exert a variety of effects on cellular physiology, growth, and differentiation. These various TGF-β isoforms share many sequence and functional homologies and identities. In contrast to many secreted proteins, TGF-β is primarily regulated at the level of post-translational activation, whereby expressed TGF-β is maintained in a latent, or inactive state, through interaction with a co-expressed protein, latency-associated peptide, or LAP. For example, the latent form of the TGF-β1 isoform is comprised of active TGF-β1 non-covalently bound to LAP. The free and latent forms of TGF-β1 and LAP are depicted in FIG. 1. In some embodiments, the TGF-β1 isoform, and its associated LAP, is the cytokine of interest for this invention. FIGS. 2-7 depict polynucleotide and corresponding protein sequences for various embodiments and isoforms of TGF-β and their associated LAPs.

In one aspect, TGF-β1 exhibits an anti-inflammatory activity. Because of this activity, TGF-β1 is useful, for example, as a pharmaceutical agent to treat individuals who have undergone solid organ transplantation. The anti-inflammatory activity of TGF-β1 aids in preventing organ rejection in the early days after transplantation. But despite its usefulness for some anti-inflammatory therapies, TGF-β1 is not an ideal anti-inflammatory agent. For example, in the instance of treatment with TGF-β1 in the context of solid organ transplantation, long-term treatment with TGF-β1 increases the risk of ultimate rejection of the transplanted organ. The reason for this effect is that TGF-β1 also exerts a fibrosis-promoting, or profibrotic activity, which can lead to rejection of the transplanted organ. Generally, the observation in the clinical setting, particularly in organ-transplant individuals, is that the predominant short-term activity of TGF-β1 is anti-inflammatory, while the predominant long-term activity is profibrotic.

In other treatment contexts, the profibrotic activity of TGF-β1 contributes to other fibrotic pathologies. For example, treatment of individuals suffering from pulmonary inflammation can lead to the pathogenesis of idiopathic pulmonary fibrosis (IPF), as well as other fibrotic diseases. More generally, endogenous TGF-β1 can lead to the development of fibrotic pathologies, particularly in individuals suffering from some types of inflammation associated with high levels of endogenous TGF-β1. TGF-β1 is the predominate isoform of TGF-β in the lung, and is a powerful profibrotic growth factor that plays an important causal role in models of lung fibrosis. TGF-β1 is found in high concentrations in the lungs of patients with pulmonary fibrosis. Accordingly, there a need for anti-inflammatory agents that are not profibrotic. There is also a need for agents which can inhibit fibrosis that is mediated by either endogenous or exogenously administered substances, such as TGF-β isoforms. In the latter instance, it is desirable that such agents can act by means other than direct binding to TGF-β, so as not to interfere with activities of TGF-β that may not be deleterious.
LAP Activities and Sequences

Latency associated peptide, in its various isoforms, is transcribed from the same gene as TGF-β in higher organisms. As shown in FIG. 1, which depicts the association between the TGF-β1 isoform and its associate LAP, LAP is known to non-covalently associate with TGF-β1. This association of these two proteins encoded by the TGF-β1 gene is referred to as the latent complex. It is understood that in the latent complex form, the biological activities of TGF-β1 are inactivated. Release or disassociation of LAP from TGF-β1 allows for TGF-β1 activation. Such dissociation may be facilitated by proteases, integrins and/or other proteins such as CD36 or thrombospondin-1.

Despite what is known about the forms and genetic encoding of LAP, its biological function and activities are not fully understood. In fact, prior to the efforts of Applicant, as first disclosed herein, the conventional understanding has been that the key function of LAP, and in particular the LAP as it is associated with TGF-β1, is as an inert scavenger of TGF-β1, binding active TGF-β1 and neutralizing its biological activity.

Applicants' studies have shown that LAP has additional activities that were previously unknown and that reveal LAP functions that are not dependent on binding of LAP to TGF-β1. These TGF-β1-binding-independent activities comprise anti-inflammatory activity without profibrotic activity, inhibition of fibrosis, and inhibition of the activities of TGF-β1.

Applicants have shown that LAP has anti-inflammatory activity, as reported herein in EXAMPLE 1. In contrast to TGF-β1, the anti-inflammatory activity of LAP is advantageous in that it is not associated with profibrotic activity.

Applicants have also shown that LAP has anti-fibrotic activity and TGF-β inhibitory activity that is achieved without binding of LAP to TGF-β. Specifically, applicants have shown that LAP inhibits the signaling activity of TGF-β1 and, specifically, that LAP inhibits the ability of TGF-β1 to increase expression of genes that contribute to fibrosis, as reported herein in EXAMPLE 2 and EXAMPLE 3. This activity of LAP is not a result of LAP binding to TGF-β. Applicant's result indicate that the LAP anti-fibrotic activity may be effective to inhibit other fibrotic processes that are not attributed to TGF-β, and thus be fully independent of the presence of TGF-β.

In one embodiment, LAP is encoded by the same gene and mRNA as TGF-β1, and the LAP protein is processed from a precursor protein that also contains TGF-β1. The genes encoding TGF-β2 and TGF-β3 are also known to have LAP moieties. In one instance, the human cDNA for TGF-β1 and LAP from TGF-β1 is shown in FIG. 2. According to the embodiment depicted in FIG. 2, TGF-β1 comprises a 390 amino acid protein, the C-terminal 112 amino acids of which (shown as boxed in the figure) comprises mature TGF-β1 (amino acids 279 to 390); LAP comprises 249 amino acids (amino acids 30 to 278); and the N-terminal amino acids 1-29 comprise a 29 amino acid signal peptide.

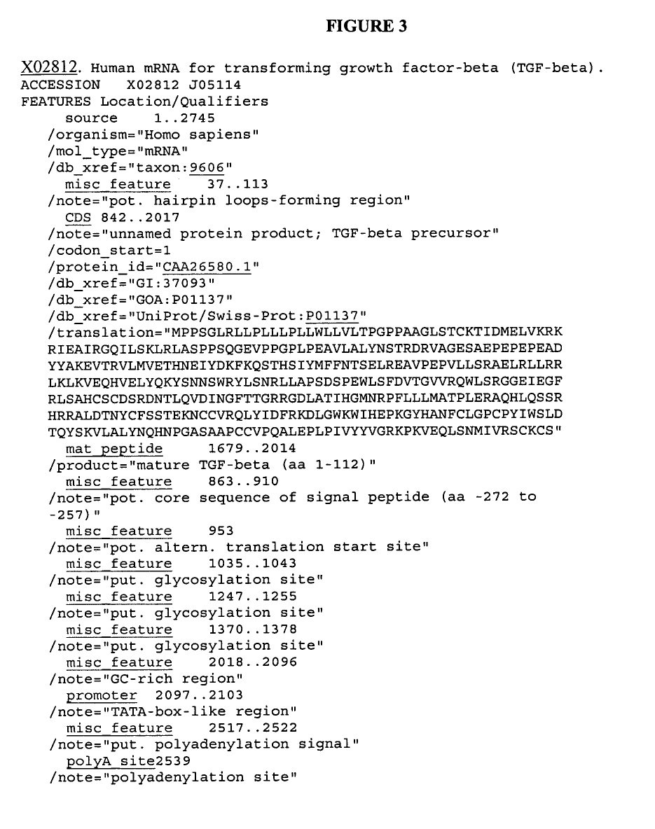

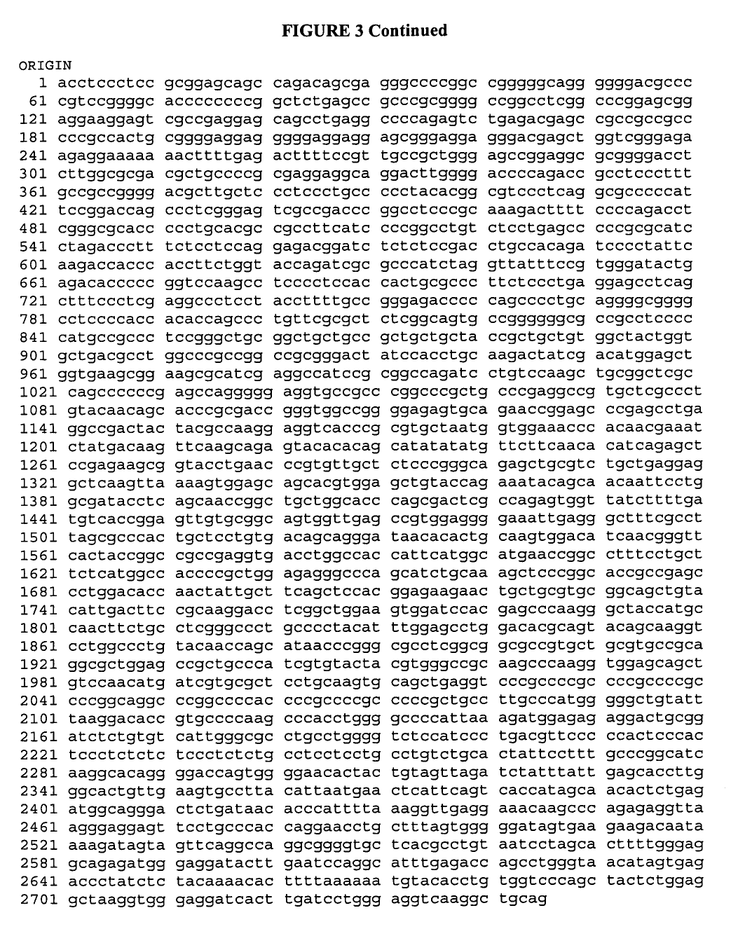

In another embodiment, TGF-β1 and LAP are encoded by a cDNA sequence as shown in FIG. 3 (said sequence corresponding to the mRNA sequence reported at GenBank Accession No. X02812 J05114). This particular mRNA has a coding sequence of 1173 bases and encodes a precursor protein of 391 amino acids in length. According to the embodiment depicted in FIG. 3, TGF-β1 comprises the C-terminal 112 amino acids of this precursor (amino acids 280 to 391). LAP comprises 250 amino acids (amino acids 30 to 279), and the signal peptide, which is generated upon cleavage from the precursor protein, comprises 29 amino acids (amino acid 1 to 29). The sequence in FIG. 3 has an Arg at amino acid position 160 of the precursor protein; this Arg is not present in the embodiment shown in FIG. 2.

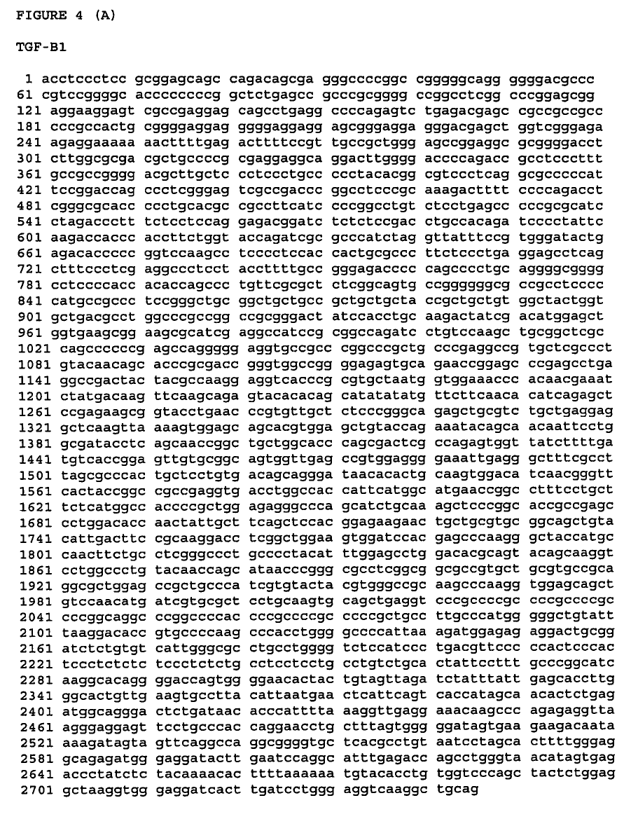

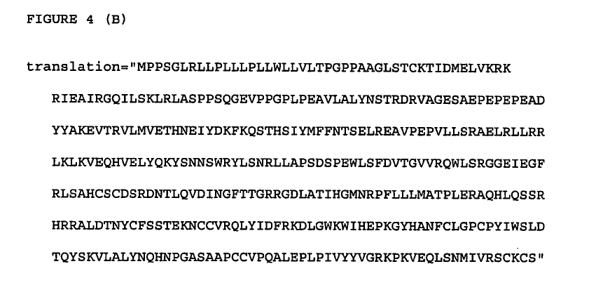

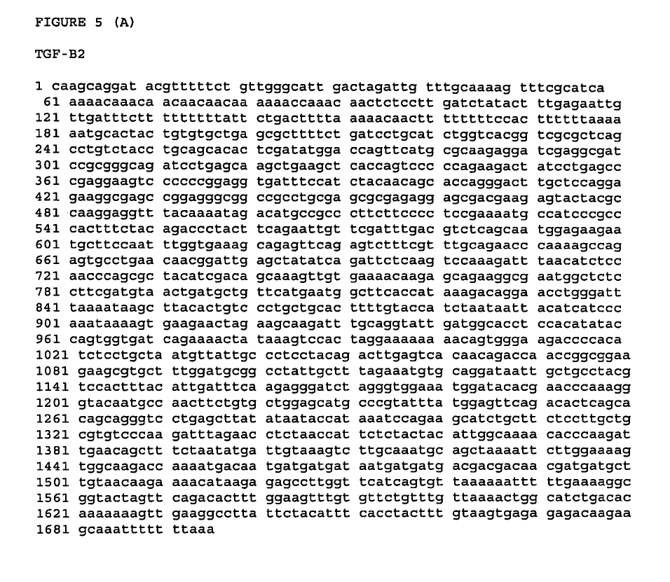

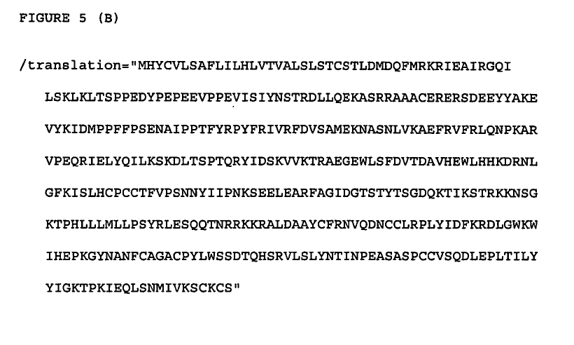

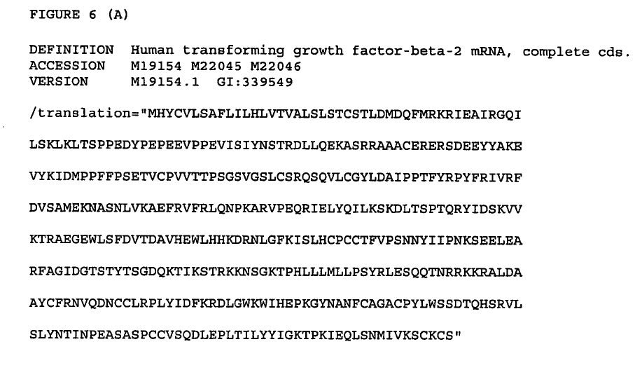

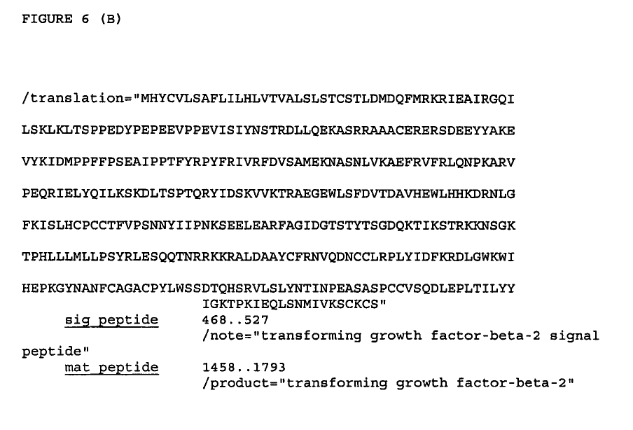

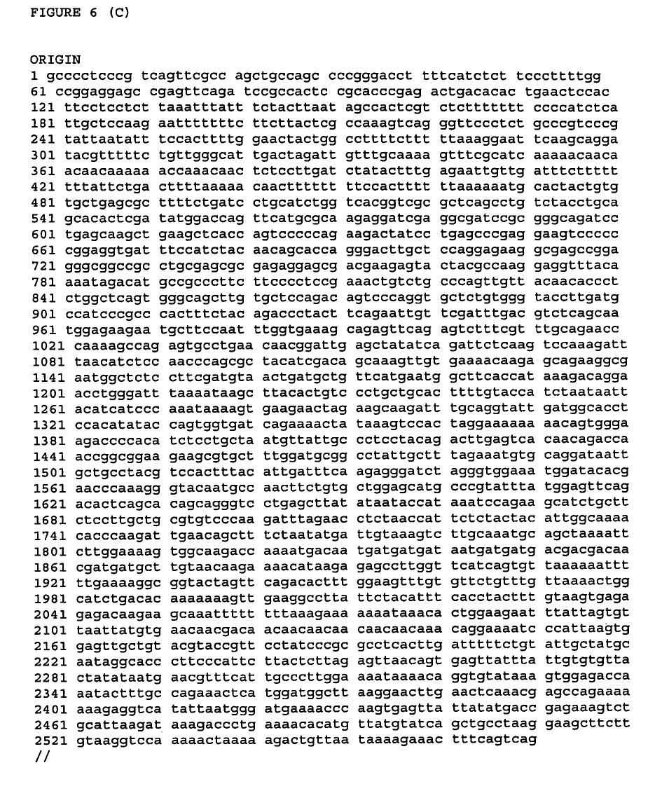

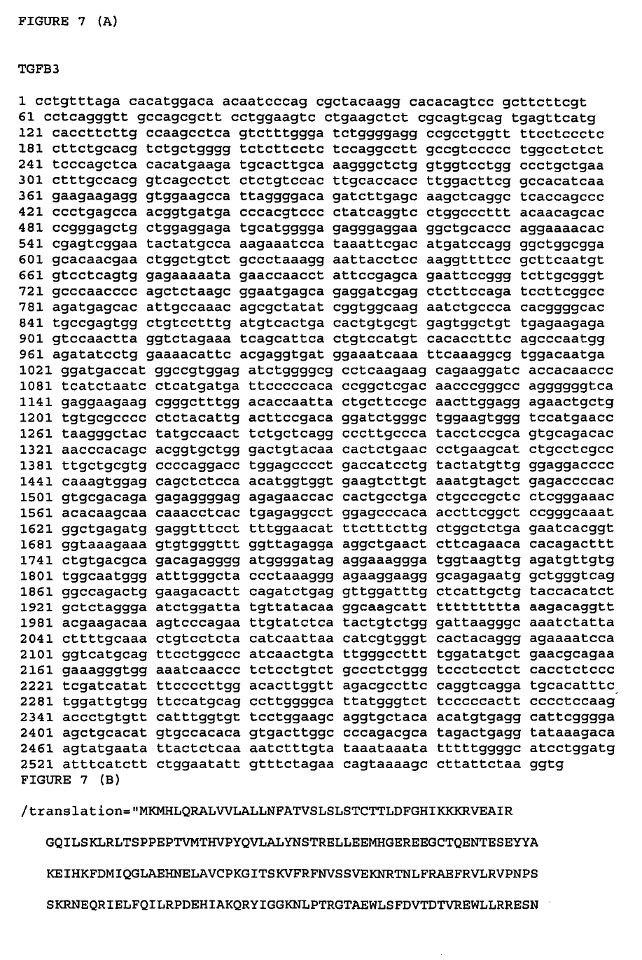

In another embodiment, TGF-β1 and LAP are encoded by a cDNA sequence as shown in FIG. 4. In yet another embodiment, the TGF-β2 isoform and LAP are encoded by a cDNA sequence as shown in FIG. 5 and FIG. 6, which sequence corresponds to that reported in GenBank under Accession Nos. M19154, M22045, and M22046. In yet another embodiment, the TGF-β3 isoform and LAP are encoded by a cDNA sequence as shown in FIG. 7, which sequence corresponds to that reported in GenBank under Accession No. NM-003239.
LAP Used in Inventive Methods

The methods and compositions of the present invention are useful for reducing or controlling inflammation or fibrosis in an individual suffering from, or at risk of developing, pathologies that include, but are not limited to, fibrotic diseases and conditions, such as pulmonary fibrosis, idiopathic pulmonary fibrosis, cirrhosis, sarcoidosis, keloids, renal fibrosis (e.g., diabetic nephropathy), retinopathy, organ transplant rejection, atherosclerosis, glomerulonephritis with scarring, cirrhosis, ARDS, fibrotic cancer, fibrosis of the lungs, arteriosclerosis, post myocardial infarction, cardiac fibrosis, post-angioplasty restenosis, renal interstitial fibrosis, scarring and diabetes-associated pathologies, and other conditions with a fibrotic component. According to the present invention, tissues including the lung, kidney, liver, and skin may be targeted, by either by systemic or local administration of various forms of LAP, to treat an individual suffering from or at risk of developing pathologies involving inflammation or fibrosis.

In some embodiments, the anti-inflammatory activity of LAP is useful for treatment of an individual suffering from or at risk of developing one or more of the diseases or pathologies listed above. Additionally, the antifibrotic activity of LAP is useful for decreasing or preventing fibrosis in such an individual. LAP is also useful for inhibiting the activities of TGF-β in such an individual. These treatments are accomplished by elevating the levels of LAP in a subject in need of the same, for example, by administering to the subject LAP protein, or a pharmaceutical composition containing LAP protein. In alternate embodiments, treatments are accomplished by elevating the levels of LAP in a subject in need of the same by administering to the subject a polynucleotide encoding LAP protein, or a pharmaceutical composition containing a polynucleotide encoding LAP protein, or a polynucleotide, protein or other agent that stimulates production of endogenous LAP protein in the subject. Preferably, LAP protein and LAP protein encoded by polynucleotides for LAP are the mature form of LAP, lacking TGF-β, and optionally lacking the signal peptide of the precursor protein. Such forms of LAP may be variants, as further discussed herein, or one of the LAP isoforms, or fragments thereof, that are associated with TGF-β isoforms. In preferred embodiments, LAP is not administered in the form of latent complex.

In some embodiments, elevation of the levels of LAP in an individual may comprise administration of a protein form of LAP, either in the form of an intact isoform or variant isoform of LAP, or a peptide or fragment thereof (referred to herein as “LAP protein”). Preferably, LAP protein is administered in its mature form, that is not in the form of the precursor protein, and not in the form of the latent complex; most preferably, LAP protein does not comprise any portions of TGF-β. When administration comprises administration of a LAP protein, the LAP protein may have one of the amino acid sequences shown in FIGS. 2, 3 and 4 of this application, that correspond to the TGF-β1 isoform of LAP. In other embodiments, the LAP protein may have other amino acid sequences, such as one of those shown in FIG. 5, 6 or FIG. 7, corresponding to the TGF-β2 and TGF-β3 isoforms of LAP. In each embodiment, the LAP protein will have one or more of the anti-inflammatory, anti-fibrotic, and anti-TGF-β activities described herein. It will be appreciated that LAP proteins derived from other members of the TGF-β superfamily (e.g., TGF-β2 and TGF-β3) may differ in their amino acid sequences, but still exhibit one or more of the anti-inflammatory, anti-fibrotic and anti-TGF-β activities described herein. Accordingly, one or more of these LAP proteins may be used in the present methods.

It will also be appreciated that certain amino acids of LAP as shown in FIGS. 2, 3 and 4, and FIGS. 5, 6 and 7 may be readily modified, substituted, or deleted without destroying the functional characteristics (i.e., anti-inflammatory, anti-fibrotic and anti-TGF-β activities) of the LAP peptide. Therefore functional analogs of LAP may be conveniently used according to the invention. Examples of such analogs include genetically or chemically modified formns of endogenous LAP. One example of an LAP modification that can be used is substitution of cysteine at position 33 of LAP to a serine. This mutation prevents the formation of disulfide linkages. Other such mutations that retain anti-inflammatory, anti-fibrotic and anti-TGF-β activities exist. Some such variants or mutants are described in international patent application WO 91/08291, the descriptions of which are incorporated herein by reference.

Other analogs are chemically synthesized compounds with similar anti-inflammatory, anti-fibrotic, and anti-TGF-β activities as LAP. Examples of such finctional analogs referred to herein also include fragments of LAP which retain the anti-inflammatory, anti-fibrotic and anti-TGF-β activities of LAP. Still other possible modifications to the LAP protein, or gene encoding the LAP protein, result in stabilization or increased half-life of the protein in the body. Still other modifications may improve the activity of the protein as compared to the activity of the endogenous protein.

In some embodiments, elevation of the levels of LAP in an individual may comprise administration of polynucleotides that encode a LAP protein. When administration of polynucleotides that encode a LAP protein are used, the polynucleotides may comprise all or a portion of the nucleic acid sequences shown in FIG. 2, FIG. 3, FIG. 4, FIG. 5 or FIG. 7 of this application, or may have other nucleic acid sequences, as long as the encoded LAP protein has one or more of the anti-inflammatory, anti-fibrotic and anti-TGF-β activities described herein. Due to the known degeneracy of the genetic code, a nucleic acid sequence encoding a LAP protein may comprise all or a portion of sequence different than that in FIGS. 2-7 and still encode an LAP protein having the amino acid sequence shown in one of FIGS. 2-7. Additionally, when polynucleotides that encode a LAP protein are used therapeutically, the nucleic acid sequence of such polynucleotides may encode one or more of the functional analogs of a LAP protein referred to above.

Preferably, the LAP protein, or polynucleotide encoding a LAP protein, that is used does not have the associated TGF-β nucleotide of amino acids sequence.
Therapeutic uses of LAP proteins and LAP protein-encoding polynucleotides

The present invention provides methods for providing to an individual one or more activities of LAP, by administering LAP to the individual. These methods may involve administration of LAP protein or nucleic acids (DNA or RNA) encoding LAP, or pharmaceutical compositions comprising LAP protein or nucleic acids. The methods also involve introduction of the LAP proteins or nucleic acids encoding LAP into an individual in the context of cells (e.g., ex vivo gene therapy). The methods also involve techniques to increase the amount of endogenous LAP in an individual.
Introduction of LAP Proteins

In some embodiments of the invention, the methods involve introducing or administering LAP proteins to individuals in need of treatment or prophylaxis for inflammation or fibrosis. There are a variety of methods well known in the art of molecular biology and biochemistry for producing and purifying proteins. Any of these can be used to prepare LAP protein suitable for administration to a subject. One example of production of recombinant LAP using baculovirus infection of insect cells is described in a publication by Munger et al., Molecular Biology of the Cell, 9:2627-2638 (1998). LAP protein is also available commercially from a variety of vendors.

In one embodiment, LAP may also be introduced into individuals in such a way that the protein does not, at least initially, find its way into cells. For example, the LAP may be introduced into an individual in such a way that the protein gets into the bloodstream of the individual. In this way, LAP may have the ability to attach to cellular receptors, if any, for LAP.

In another embodiment, LAP may be introduced into individuals in such a way that the protein is able to get into cells. A variety of methods exist for introducing proteins into cells. In one method, proteins are coupled or fused to short peptides that direct entry into cells. One such group of peptides are called protein transduction domains. Another method for introducing proteins into cells uses lipid carriers. For example, proteins that are associated with liposomes are able to enter cells when the liposomes enter or fuse with cells. Other methods of introducing proteins into cells are known. Microinjection and electroporation are two such methods. Other methods are known.

Preferably, LAP introduced into an individual in protein form is recombinant LAP which can be made and purified using a variety of methods as previously discussed. Preferably, the LAP protein is part of a pharmaceutical composition.
Introduction of LAP-Encoding Polynucleotides

In one embodiment of the invention, the methods involve introducing polynucleotides that encode LAP into an individual. Introduction of such LAP-encoding polynucleotides can be achieved using a variety of methods. Many of these methods are well known in the art of gene therapy. For example, in some embodiments, polynucleotides can be introduced by transfection or infection with viral vectors encoding LAP. LAP-encoding DNA can be amplified using any of various recombinant DNA methodologies well known in the art.

In one embodiment, the invention provides for introduction of LAP-encoding polynucleotides into cells that are present within an individual. In order to introduce the LAP-encoding polynucleotides into cells, the protein coding region of the polynucleotide is normally attached to sequences that facilitate its transcription into mRNA (if the nucleic acid is DNA) as well as translation of the mRNA into protein. A strategy common in the art for doing this is to clone the polynucleotide sequence encoding LAP into a vector which contains sequences facilitating expression of a protein coding sequence cloned therein.

Expression vectors normally contain sequences that facilitate gene expression. An expression vehicle can comprise a transcriptional unit comprising an assembly of a protein encoding sequence and elements that regulate transcription and translation. Transcriptional regulatory elements generally include those elements that initiate transcription. Types of such elements include promoters and enhancers. Promoters may be constitutive, inducible or tissue specific. A variety of promoters that are expressed in specific tissues exist and are known in the art. For example, promoters whose expression is specific to neural, liver, epithelial and other cells exist and are well known in the art. Transcriptional regulatory elements also include those that terminate transcription or provide the signal for processing of the 3′ end of an RNA (signals for polyadenylation). Translational regulatory sequences are normally part of the protein encoding sequences and include translational start codons and translational termination codons. There may be additional sequences that are part of the protein encoding region, such as those sequences that direct a protein to the cellular membrane, a signal sequence for example. Methods for making such DNA molecules (i.e., recombinant DNA methods) are well known to those skilled in the art.

In the art, vectors refer to nucleic acid molecules capable of mediating introduction of another nucleic acid or polynucleotide sequence to which it has been linked into a cell. One type of preferred vector is an episome, i.e., a nucleic acid capable of extrachromosomal replication. Other types of vectors become part of the genome of the cell into which they are introduced. Vectors capable of directing the expression of inserted DNA sequences are referred to as “expression vectors” and may include plasmids, viruses, or other types of molecules known in the art. One preferred type of vector is a recombinant virus that contains a cloned LAP-encoding nucleic acid. The virus is administered to the individual where it infects the desired cells and produces the LAP. Another method for introducing LAP-encoding nucleic acids involves administration of purified DNA or RNA encoding LAP directly into the individual. Such administration can be done by injection of the nucleic acid.

Viral vectors are recombinant viruses which are generally based on various viral families comprising poxviruses, herpesviruses, adenoviruses, parvoviruses and retroviruses. Such recombinant viruses generally comprise an exogenous polynucleotide sequence (herein, the LAP gene) under control of a promoter which is able to cause expression of the exogenous polynucleotide sequence in vector-infected host cells.

One type of viral vector is a defective adenovirus which has the exogenous polynucleotide sequence inserted into its genome. The term “defective adenovirus” refers to an adenovirus incapable of autonomously replicating in the target cell. Generally, the genome of the defective adenovirus lacks the sequences necessary for the replication of the virus in the infected cell. Such sequences are partially or, preferably, completely removed from the genome. To be able to infect target cells, the defective virus contains sufficient sequences from the original genome to permit encapsulation of the viral particles during in vitro preparation of the construct. Other sequences that the virus contains are any such sequences that are said to be genetically required “in cis.”

Another type of viral vector is a defective retrovirus which has the exogenous polynucleotide sequence inserted into its genome. Such recombinant retroviruses are well known in the art. Recombinant retroviruses for use in the present invention are preferably free of contaminating helper virus. Helper viruses are viruses that are not replication defective and sometimes arise during the packaging of the recombinant retrovirus.

Non-defective or replication competent viral vectors can also be used. Such vectors retain sequences necessary for replication of the virus.

Typically, vectors contain one or more restriction endonuclease recognition sites which permit insertion of the LAP polynucleotide sequence. The vector may further comprise a marker gene, such as for example, a dominant antibiotic resistance gene, which encode compounds that serve to identify and separate transformed cells from non-transformed cells.

In one aspect, the present methods comprise introduction of LAP-encoding polynucleotides, preferably contained within a vector, into specific cells so that the cells have increased levels of LAP. Herein, such introduction or transfer of a DNA molecule or molecules, specifically a DNA molecule encoding one or more LAP-encoding sequences, into a cell refers to any of a variety of methods known in the art to get DNA molecules into cells. One such method, whereby isolated DNA is introduced into cells, is know as transfection. Such transfection is commonly performed using various treatments of the cells or DNA which facilitate uptake of the DNA by the cell. For example, cells can be treated chemically to make them permeable to DNA. DNA can also be treated, for example by containing the DNA within liposomes that cells can internalize. Preferably, transfection is used to introduce plasmid DNA into cells.

As described above, LAP-encoding polynucleotide sequences can also be introduced into cells using viruses. For example, polynucleotide sequences that are to be introduced into cells are cloned into viral genomes. Infection of cells with such viruses results in introduction of the viral genome into the cell. Since the cloned polynucleotide sequence is part of the viral genome, it is introduced into the cell along with the viral genome. Such viral “vectors” can have DNA or RNA genomes. Numerous such viral vectors are well known to those skilled in the art. Viral vectors that have cloned polynucleotide sequences, encoding LAP proteins for example, cloned into their genomes are referred to as “recombinant” viruses. Transfer of DNA molecules using viruses is particularly useful for transferring polynucleotide sequences into particular cells or tissues of an animal. Such techniques are commonly known in the art as gene therapy.

Whatever methodology is used to administer the LAP-encoding nucleic acids to individuals, such methodologies may comprise variations that result in the LAP-encoding nucleic acids being preferentially introduced into certain desired cells (e.g., lung cells in the case of pulmonary fibrosis). For example, techniques are known in the art that result in recombinant viruses specifically infecting certain cell types within a human or animal. For viruses, such “targeting” can be accomplished through manipulation of cellular receptors for the recombinant viruses and/or manipulation of viral ligands that recognize and bind to cellular receptors for the viruses.

After LAP polynucleotide sequences are introduced into cells, techniques are used to determine specifically the cells into which the polynucleotide sequences have been introduced and/or the specific cells that are expressing the introduced polynucleotide sequences. A variety of techniques to examine the presence of polynucleotide sequences and/or expression of polynucleotide sequences exist and are well known in the art. Some such techniques include Southern blotting, Northern blotting, polymerase chain reaction (PCR), Western blotting, RNase protection, radioiodide uptake assays, and others.
Pharmaceutical Compositions

The LAP-encoding polynucleotides, LAP proteins and the like are preferably formulated into pharmaceutical compositions. Suitable formulations for delivery are found in Remington's Pharmaceutical Sciences, 17th ed. (Mack Publishing Co., Philadelphia, Pa., 1985). These pharmaceutical compositions are suitable for use in a variety of drug delivery systems (Langer, Science 249:1527-1533, 1990).

The LAP polypeptides or proteins may be prepared with generally used diluents, excipients, vehicles and additives such as filler, extender, binder, carrier, salt, moisturizing agent, disintegrator, disintegrator retarder, absorption promoters, adsorbent, glidant, buffering agent, preservative, dispersing agent, wetting agent, suspending agent, surfactant, lubricant and others. The LAP polynucleotides or proteins may have a variety of dosage forms depending on their therapeutic purpose; typically tablet, pill, powder, solution, suspension, emulsion, granule, capsule, injection (e.g., solution, suspension) and suppository.

Injection, solution, emulsion and suspension forms of the LAP polynucleotides or proteins are sterilized and preferably isotonic with blood. Such forms may be prepared using diluents commonly used in the art; for example, water, ethanol, macrogol, propylene glycol, ethoxylated isostearyl alcohol, polyoxyisostearyl alcohol and polyoxyethylene sorbitan fatty acid esters. The pharmaceutical preparation may contain sodium chloride necessary to prepare an isotonic solution, glucose or glycerin, as well as usual solubilizers, buffers and soothing agents.

Compositions suitable for parenteral administration conveniently comprise a sterile, pyrogen-free, aqueous or oleaginous preparation of the LAP nucleic acids or proteins which are preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. The pharmaceutical compositions may conveniently be presented in unit dosage form and may be prepared by any of the methods well-known in the art of pharmacy.

Additionally, preparation of parenterally-acceptable solutions of the pharmaceutical composition, having due regard to pH, isotonicity, stability, and the like, is within the level of ordinary skill in the art of pharmacy and pharmacology. A preferred pharmaceutical composition for injection can contain, in addition to the vector, an isotonic vehicle such as Sodium Chloride Injection, Ringer's Injection, Dextrose Injection, Dextrose and Sodium Chloride Injection, Lactated Ringer's Injection, phosphate buffered saline (PBS), or other vehicle as known in the art. The pharmaceutical composition used in the methods of the present invention may also contain stabilizers, preservatives, buffers, antioxidants, or other additives known to those of skill in the art.
Administration of LAP to Individuals

A biologically effective amount of the LAP protein or polynucleotide is administered to the individual. Herein, a biologically effective amount of LAP is an amount sufficient to produce the desired effect, e.g., reduced fibrosis, inhibition of TGF-β activity, or reduced inflammatory activity.

The biologically effective amount is administered to the individual as a single dose, but more likely as a series of dosages over a period of days, weeks or even months. Herein, an effective therapeutic dose is a dose that accomplishes the desired result.

Dose of the LAP may be selected, depending on their dosage form, individual's age, sex and severity of disease, and other conditions, as appropriate, but the amount of the active ingredient may be generally about 0.0001 to 100 mg/kg a day. It is recommended that a unit dosage form may contain about 0.001 to 1000 mg of the active ingredient.

The LAP, generally speaking, may be administered using any mode that is medically acceptable, meaning any mode that produces effective levels of the active LAP without causing clinically unacceptable adverse effects. Such modes of administration include parenteral routes (e.g., intravenous, intra-arterial, subcutaneous, intramuscular, mucosal or infusion), but may also include oral, rectal, topical, nasal or intradermal routes. Another route of introduction, of special use for treatment of individuals with pulmonary fibrosis, is the respiratory route by inhalation into the lungs. Other delivery systems can include time-release, delayed release or sustained release delivery systems. Such systems can avoid repeated administrations, increasing convenience to the individual and the physician. Many types of release delivery systems are available and known to those of ordinary skill in the art.

The pharmaceutical compositions of the present invention may also be administered by the respiratory route. The formulations administered by the respiratory route are generally oral aerosol formulations. Such formulations can be administered via the respiratory route in a variety of ways.

In the event that a response in an individual is insufficient at the initial doses applied, higher doses (or effectively higher doses by a different, more localized delivery route) may be employed to the extent that individual tolerance permits. Multiple doses per day are contemplated to achieve appropriate systemic levels of peptides.

The duration of therapy with the pharmaceutical compositions used in the methods of the present invention will vary, depending on the unique characteristics of the pharmaceutical composition and the particular therapeutic effect to be achieved, the severity of the disease being treated and the condition and potential idiosyncratic response of each individual. Ultimately the attending physician will decide on the appropriate duration of therapy with the pharmaceutical composition used in the method of the present invention.

Preferably, the methods of treatment described herein result in the individual being cured of the particular condition that is being treated. However, effects of the inventive treatment may also be measured as an improvement in the quality of life of the individual.
Dosage Determination

LAP is administered to the host subject in a biologically effective amount. As used herein, the term “biologically effective amount” means the total amount of LAP that is sufficient to show a meaningful benefit, i.e., treatment, healing, prevention, amelioration, or reduction in the symptoms of inflammation or fibrosis or an increase in rate of healing, amelioration or reduction in the symptoms of conditions.

By “treating” is meant curing or ameliorating the inflammation or inflammation associated fibrosis or tempering the severity of the condition. By preventing is meant blocking the formation of fibrosis.

Appropriate dosages can be determined in view of this disclosure by one of ordinary skill in the art by running routine trials with appropriate controls. Initial studies to determine appropriate dosage ranges are conducted in cells and in animal model systems. For example, studies to determine appropriate dosages for reducing inflammation can be conducted in apo E deficient mice. Such initial studies include determining a dosage range that reduces signaling in myeloid cells stimulated with M-CSF or that reduces fibrosis in Bleomycin-treated C57B1/6 mice. Additional assays include determining a dosage that blocks TBFP induced Smad nuclear translocation and luciferase production in TMLC cells, i.e., mink lung epithelial cells stably transfected with a TGFP sensitive promoter region that drives luciferase production.

Appropriate dosage ranges are further optimized in clinical studies on patients in need of such treatment, e.g. patients with tissue repair diseases such as pulmonary fibrosis, fibrotic renal diseases, arthritis, cardiomyopathy, fibroproliferative ARDS or patients with allogeneic problems such as transplantation or patients with diseases associated with TGFB, such as cancer. In such studies, the effective dose may be determined by monitoring organ function, e.g. PFTs for lung, joint mobility for arthritis, echocardiogram for heart, tumor size for cancer, or organ function for transplant patients. Comparison of the appropriate treatment groups to the controls will indicate whether a particular dosage is effective in preventing or treating the infection at the levels used in a controlled challenge.

### EXAMPLES

The invention may be better understood by reference to the following examples, which serve to illustrate but not to limit the present invention.

### Example 1

### Anti-inflammatory Activity of LAP

A study was done to determine if LAP had in vivo anti-inflammatory properties. The study determined whether LAP could inhibit a delayed type hypersensitivity reaction (DTHR), a type of cellular mediated immune reaction. The assay used involved transfer of syngeneic splenocytes from cardiac allograft rejector mice DBA/2 to C57B1/6 recipients (DBA/2→ C57B1/6 transfer) between 30-60 days post transplant. For this assay, syngeneic splenocytes from the transplanted mice, plus subcellular DBA/2 alloantigen, were injected into the pinnae of recipient mice. The assay also included injections as above, also including 5 ng of porcine TGF-β or 10 pg of human LAP. Changes in ear thickness were measured both before injection and 24 hours after injection using a dial thickness gauge (Swiss Precision Instruments) as a measure of DTHR.

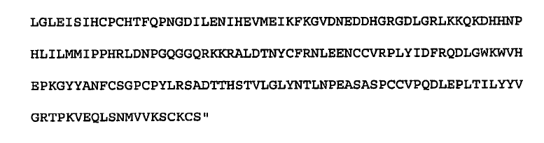

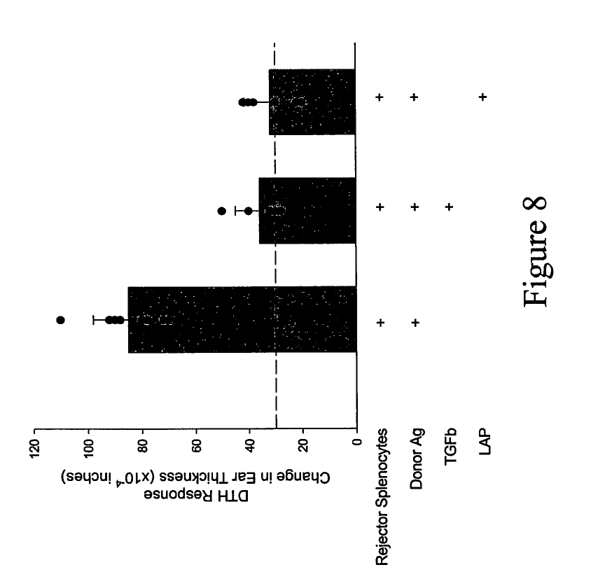

As is known to occur, the transfer caused a DTHR as indicated by the increase in ear thickness of the mice. As is also known, TGF-β reduced the ear inflammation to give an acceptor phenotype (FIG. 8). The results also showed that recombinant human LAP functioned as well as TGF-β to reduce ear inflammation. The data are the mean +/− SD of five independent studies. The data show that LAP functioned as an anti-inflammatory agent.

In another study, it was shown that antibodies specific for TGF-β inhibited the effect of TGF-β on the reduction of ear inflammation but did not affect the effect of LAP on the reduction of ear inflammation in this system. Antibodies specific for LAP inhibited the effect of LAP on the reduction of ear inflammation but did not affect the effect of TGF-β on the reduction of ear inflammation in this system. These data demonstrate that the anti-inflammatory activity of LAP works through a mechanism independent of TGF-β.

### Example 2

### LAP inhibition of the Signaling Activity of TGF-β

A study was done to determine if LAP inhibited the signaling activity of TGF-β, independent of binding and sequestering TGF-β, as shown in FIG. 1. It is known that when active TGF-β signals through TGF-β receptors, certain transcriptional promoters are regulated. One such promoter is the promoter regulating plasminogen activator inhibitor-1, a protein that promotes fibrin blood clot formation or inhibits fibrin clot dissolution. In the study, TMLC cells were used. TMLC cells are transformed mink lung epithelial cells that contain plasminogen activator inhibitor-1 promoter elements linked to a luciferase reporter gene. In these cells, TGF-β causes increases in luciferase gene expression and inhibition of TGF-β signaling activity causes decreases in luciferase gene expression.

In this study, TMLC cells were incubated with: recombinant human TGF-β1 (1 ng/ml) alone; or LAP (250 ng/ml) alone; or preincubated with LAP (250 ng/ml), washed and then incubated with TGF-β1 (1 ng/ml); or preincubated with LAP (250 ng/ml) and then incubated with TGF-β1 (1 ng/ml) for 18 hours. The cells were then harvested, lysed and luciferase activity was determined using a luminometer. Washing of the cells and elimination of LAP, as in the third experiment above, was done to eliminate the possibility that LAP that was present would bind to and inactivate added TGF-β.

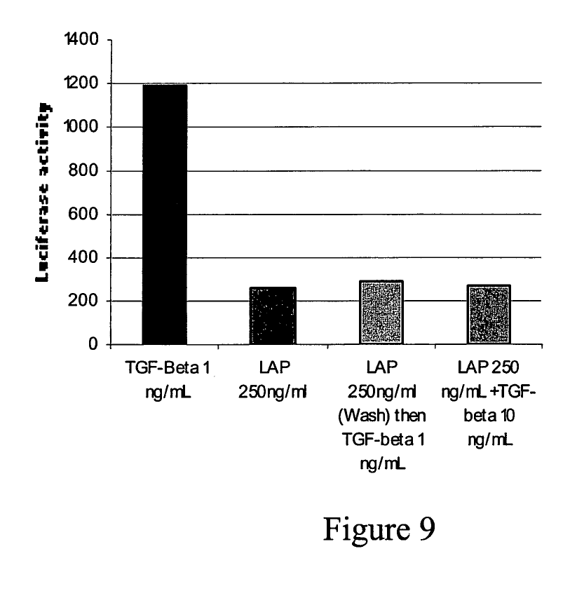

The data (FIG. 9) are representative of two independent studies. The data showed that preincubation with LAP followed by washing, blocked the ability of TGF-β to stimulate expression of the plasminogen activator inhibitor-1 promoter. Additionally, preincubation of cells with LAP inhibited the ability of TGF-β to stimulate expression of the plasminogen activator inhibitor-1 promoter. These data showed that LAP reduced the biological activity of active TGF-β by a mechanism other than merely acting as a binding and sequestering agent for TGF-β.

### Example 3

### LAP Inhibition of Signaling Activities of TGF-β That Contributes to Profibrotic Activity

A study was done to determine if LAP inhibited the signaling activity of TGF-β that resulted in stimulation of expression of specific extracellular matrix materials that contribute to the profibrotic activity of TGF-β. In this study, the ability of LAP to inhibit TGF-β activation of hydroxyproline, a component of collagen, was tested.

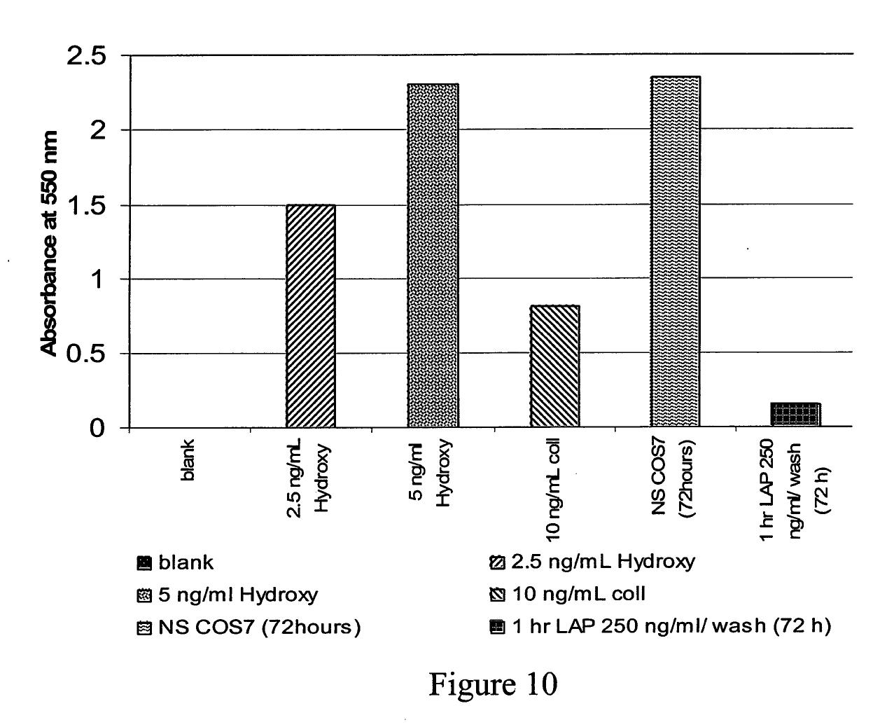

In this study, COS-7 cells were suspended in 10% fetal calf serum as a source of TGF-β. The cells were then spiked with either vehicle control alone for 72 hours (see lane labeled “NS COS7” in FIG. 10); or with 250 ng/ml recombinant LAP for 1 hour, the cells washed, then replaced with media containing 10% fetal calf serum for 72 hours (see lane labeled “1 hr LAP 250 ng/ml/ was (72 h)” in FIG. 10. The cells were then harvested and assayed for hydroxyproline as described in the detailed methods.

1000 μl of supernatants from the cells were placed in 1.5 ml Eppendorf tubes. They were dried at 60° C. in a fume hood overnight. The pellet was re-suspended in 40 μl of PBS, and mixed thoroughly. Ten μl of 10M NaOH was added. Tubes were then left open and heated in an autoclave for 20 min at 121° C. Standards of hydroxyproline were made ranging from 0 to 500 μg in 50 ml 2M NaOH. Collagen standards were similarly made. To the standards was added 450 μl of chloramine-T solution (1.27 chloramine T in 20 ml 50% n-propanol and brought to 100 ml in Acetate-Citrate buffer (120 g sodium acetate trihydrate, 46 g citric acid, 12 ml acetic acid, and 34 g sodium hydroxide in 1 liter of distilled water) is added. Tubes were then incubated for 25 minutes at room temperature. 500 μl of freshly made Ehrlich's reagent (1.5 g p-dimethylaminobenzaldehyde in 8 ml n-propanol and 4 ml perchloric acid) was added, tubes were closed and mixed, and heated at 60° C. for 20 minutes. Absorbance was measured at 550 nm.

The data showed that LAP reduced hydroxyproline production and levels of collagen (FIG. 10). Controls to show that the assay itself was functioning were assays of hydroxyproline (2.5 ng/ml or 5 ng/ml) and collagen (10 ng/ml). This figure is representative of two independent studies. The indication is that, not only does LAP not have profibrotic activity, but that it inhibits profibrotic activity, especially the profibrotic activity of TGF-β.

The disclosure of all patents, patent applications (and any patents which issue thereon, as well as any corresponding published foreign patent applications), GenBank and other accession numbers and associated data, and publications mentioned throughout this description are hereby incorporated by reference herein. It is expressly not admitted, however, that any of the documents incorporated by reference herein teach or disclose the present invention.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While particular embodiments of the subject invention have been described, it will be obvious to those skilled in the art that various changes and modifications of the subject invention can be made without departing from the spirit and scope of the invention. In addition, while the present invention has been described in connection with certain specific embodiments thereof, it is to be understood that this is by way of illustration and not by way of limitation and the scope of the invention is defined by the appended claims which should be construed as broadly as the prior art will permit.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for controlling inflammation in a subject in need of the same, comprising, increasing the biologically active level of latency-associated peptide (LAP) in the subject.

2. The method according to claim 1, wherein the increased the biologically active level of LAP does not promote fibrosis in the subject.

3. The method according to claim 1, wherein the increased the biologically active level of LAP is achieved by administering to the subject an expression vector which encodes LAP protein.

4. The method according to claim 1, wherein the increased the biologically active level of LAP is achieved by administering to the subject LAP protein.

5. A pharmaceutical composition comprising LAP protein and one or more components selected from the group consisting of, excipients, vehicles, additives, and other therapeutic agents.

6. A pharmaceutical composition comprising an expression vector encoding LAP protein and one or more components selected from the group consisting of, excipients, vehicles, additives, and other therapeutic agents.

7. A method for reducing or inhibiting fibrosis in a subject in need of the same, comprising, increasing the biologically active level of LAP in the subject.

8. The method according to claim 7, wherein the increased the biologically active level of LAP is achieved by administering to the subject an expression vector which encodes LAP protein.

9. The method according to claim 7, wherein the increased the biologically active level of LAP is achieved by administering to the subject LAP protein.

10. The method according to claim 7, wherein said subject has received a solid organ transplant.

11. The method according to claim 7, wherein the subject exhibits symptoms of, or is predisposed to developing, pulmonary fibrosis.

12. A method for inhibiting a delayed-type hypersensitivity reaction in a subject who has received a solid organ transplant comprising, increasing the biologically active level of LAP in the subject.

13. The method according to claim 12, wherein the increased the biologically active level of LAP is achieved by administering to the subject an expression vector which encodes LAP protein.

14. The method according to claim 12, wherein the increased the biologically active level of LAP is achieved by administering to the subject LAP protein.

15. A method for reducing the profibrotic activity of TGFβ in a subject, increasing the biologically active level of LAP in the subject.

16. The method according to claim 15, wherein the increased the biologically active level of LAP is achieved by administering to the subject an expression vector which encodes LAP protein.

17. The method according to claim 15, wherein the increased the biologically active level of LAP is achieved by administering to the subject LAP protein.

18. The method according to claim 15, wherein the subject has been treated with TGFPβ1.

19. The method according to claim 15, wherein the subject has exhibited symptoms of inflammation.

20. A method for inhibiting TGFβ1 induced signaling activity in cells that have been exposed to TGFβ1, comprising: contacting said cells with LAP.

21. The method according to claim 21, wherein the cells are contacted with LAP by administration to the cells of an expression vector which encodes LAP protein.

22. The method according to claim 21, wherein the cells are contacted with LAP protein.
